(19) United States
(12) Patent Application Publication
(10) Pub. No.: US 2006/0233849 A1
(43) Pub. Date: Oct. 19, 2006

(54) Composite bone graft material

(76) Inventors: Bruce J. Simon, Mountain Lakes, NJ (US); Robert M. Ronk, Pierceton, NJ (US); Paul D' Antonio, Morristown, NJ (US); Jeffrey D. Schwardt, Palo Alto, CA (US)

Correspondence Address: HARNESS, DICKEY & PIERCE, P.L.C., P.O. BOX 828, BLOOMFIELD HILLS, MI, 48303, US

(21) Appl. No.: 11/105,334

(22) Filed: Apr. 13, 2005

(51) Int. Cl. A61F 13/00

(52) U.S. Cl. 424/422

(57) ABSTRACT

A bone graft material comprising about 50-90% quickly bioresorbable porogen particles and about 10-50% of a calcium phosphate compound or salt matrix material. A bioactive substance may be included in the matrix material, the porogen particles, or both. Commercial packages containing the bone graft materials and methods for repairing bone therewith are also claimed.

### BACKGROUND

The present invention involves the field of bone graft materials. A multiplicity of bone graft materials has been provided in the art for repairing defects in bone, including materials for adhering bone graft and implants to bone surfaces. These typically have taken the form of calcium phosphate-based, or gel-based materials. In order to enhance the rate of resorption of such materials, porous forms of these materials have been created. In many cases, this involves administration of a bone graft material that contains a significant proportion of empty pores, with the concomitant risk of friability, the bone graft being brittle and subject to fragmentation. In some cases, biodegradable porogen particles have been used. However, the selection of materials and sizes for porogen particles often results in formation of pores too small for osteoblast colonization, or pores that take unduly long to form by in vivo biodegradation of the porogen, thus interfering with an efficient healing process. In some instances, porogens are used, but at such a low percentage (e.g., 20-50%) that efficient resorption of the, e.g., calcium phosphate or other matrix material is delayed. Thus, there is still a need for improved bone graft materials to speed the healing process, while providing for minimal load capability. SUMMARY

The present invention provides an improved bone graft material comprising a calcium matrix material and quickly resorbable porogen particles, the composition containing from about 50% to about 90% by volume porogen particles; and optionally containing bioactive substance(s).

The present invention further provides:

Bone graft materials having a calcium matrix component, comprising calcium phosphate compounds(s) and salt(s), and having porogen particles, in a porogen particle-to-matrix material ratio of 1:1 to about 9:1;

Such bone graft materials in which the porogen particles comprise osteoinductive demineralized bone matrix; such materials in which the porogen particles comprise biocompatible, biodegradable polymer(s); such materials in which the porogen particles comprise biocompatible, biodegradable polymer(s) having a weight average molecular weight of about 2,000 to about 100,000;

Such bone graft materials in which the porogen particles comprise at least one bioactive agent; such materials in which the porogen particles include at least one morphology that is substantially regular polyhedral, lenticular, ovate, or spherical; such materials in which the porogen particles have one or more or all of their axial, transverse, or lateral dimensions in the range from about 100 to about 500 microns; such materials in which the porogen particles have a ratio of average width to average length that is from about 5:1 to about 1:5;

Such bone graft materials in which the porogen particles are solid, hollow, or laminate particles; such bone graft materials in which the porogen particles, or at least one wall or layer thereof, are capable of biodegradation in vivo in about 10 minutes to about 8 weeks.

Such bone graft materials that are in the form of a paste, injectible solution or slurry, dry powder, or dry solid; such bone graft materials that are in the form of a paste, injectible solution or slurry that has been hydrated by application of a biological fluid to a dry powder or dry solid bone graft material;

Commercial packages containing such a bone graft material and instructions for use thereof in repairing bone; and

Methods for repairing bone by providing such a bone graft material and administering it to a living bone tissue surface in need thereof; such methods further comprising permitting the material to remain at an in vivo site in which it is placed, for a sufficient time to permit porogen particles thereof to be biodegraded in vivo.

It has been discovered that compositions and methods of this invention afford advantages over bone graft materials known in the art, including one or more of enhanced rates of integration, calcium phosphate matrix resorption, and osteoblast colonization. Further uses, benefits and embodiments of the present invention are apparent from the description set forth herein.

### DETAILED DESCRIPTION

Glossary

The following definitions and non-limiting guidelines must be considered in reviewing the description of this invention set forth herein. The headings (such as “Introduction” and “Summary,”) and sub-headings (such as “Compositions” and “Methods”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., as being a “system”) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features. Specific Examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “about,” when applied to the value for a parameter of a composition or method of this invention, indicates that the calculation or the measurement of the value allows some slight imprecision without having a substantial effect on the chemical or physical attributes of the composition or method.

The term “a” as used herein means at least one.

As used herein, the word “include,” and its variants, is intended to be non-limiting, such that recitation of items in a list is not to the exclusion of other like items that may also be useful in the materials, compositions, devices, and methods of this invention.

Although the open-ended term “comprising,” as a synonym of terms such as including, containing, or having, is use herein to describe and claim the present invention, the invention, or embodiments thereof, may alternatively be described using more limiting terms such as “consisting of” or “consisting essentially of” the recited ingredients. Thus, for example, in a preferred embodiment, a bone graft material according to the present invention comprises a combination of about 10-25% of a calcium-based matrix material and about 75-90% by volume biodegradable porogen particles, but the composition may contain almost solely those two components, or may consist or consist essentially of those components.

Bone Graft Materials

A bone graft material according to the present invention will comprise a calcium-based matrix material and porogen particles, as further defined below, the ratio of porogen particles to matrix material being from about 1:1 to about 9:1. The matrix may, in addition, contain other substances that collectively comprise about 10% by volume or less of the matrix, preferably about 5% or less, about 3%, about 2% or about 1% or less of the matrix. The porogen particles will preferably make up about 50% to about 90% by volume of the bone graft material; preferably about 75% to about 90%. The matrix material component makes up the remainder. In a preferred embodiment, the porogen particles will be susceptible to biodegradation within about 10 minutes to about 8 weeks; preferably about 10 minutes to about 6 weeks, about 10 minutes to about 4 weeks, about 10 minutes to about 2 weeks, or about 10 minutes to about 1 week.

A bone graft material according to the present invention provides an osteoinductive scaffold for promoting bone healing, as well as for enhanced colonization by osteoblasts, even without use of expensive osteoinductive factors in the porogen particles, which factors are thus optional therein. The material can support a minimal load. Further advantages of the composition may include: ease and economy of manufacturing, increased proportion of porogen particles and enhanced biodegradation rate of the selected porogen material enhances development of in vivo porosity to expedite bone cell colonization, increase the rate of calcium phosphate matrix resorption, and decrease the time needed for healing; the ability to wet or suffuse the composition with autologous biological fluids to thereby further enhance the healing properties of the material; the lack or reduced frequency of empty pores in the material as administered can reduce the immediate potential for the material to be friable as a result of different resorption rates of component materials.

A bone graft material according to the present invention may be provided in the form of a bone paste, a shaped solid, or a dry pre-mix useful for forming such a paste or solid. The phrase “bone paste” refers to a slurry or semi-solid composition of any consistency that hardens to form a solid structure, and thus includes, e.g., bone plasters, putties, adhesives, cements, bone void fillers, and bone substitutes. As a result, the bone paste can be any composition capable of being injected, molded, painted, suffused, or placed into contact with a bone surface in vivo. The “shaped solid” may take any form, including a pellet that can be placed into a bone void or into contact with a bone surface in vivo. The dry pre-mix may be provided in the form of a powdered and/or granular material.

Calcium Matrix Component

A calcium matrix component (CMxC) for use herein will include one or more of the following calcium phosphate compounds and salts, and combinations thereof:
tricalcium phosphate Ca₃(PO₄)₂ (TCP), including alpha-TCP, beta-TCP, and biphasic calcium phosphate containing alpha- and beta-TCP;
amorphous calcium phosphate (ACP);
monocalcium phosphate Ca(H₂PO₄)₂ (MCP) and monocalcium phosphate monohydrate Ca(H₂PO₄)₂.H₂O (MCPM);
dicalcium phosphate CaHPO₄ (DCP) and dicalcium phosphate dihydrate CaHPO₄.2H₂O (DCPD);
tetracalcium phosphate Ca₄(PO₄)₂O (TTCP);
octacalcium phosphate Ca₈(PO₄)₄(HPO₄)₂.5H₂O (OCP);
calcium hydroxyapatite Ca₁₀(PO₄)₆(OH)₂ (CHA);
calcium oxyapatite Ca₁₀(PO₄)₆O (COXA);
calcium carbonate apatite Ca₁₀(PO₄)₆CO₃ (CCA);
calcium carbonate hydroxyapatites, e.g., Ca₁₀(PO₄)₅(OH)(CO₃)₂ and Ca₁₀(PO₄)₄(OH)₂(CO₃)₃ (CCHA);
calcium-deficient calcium phosphates in which the molar or mass ratio of Ca:P is reduced by about 20% or less, preferably about 15% or less, preferably about 10% or less, relative to the corresponding calcium-non-deficient species, examples of which include: calcium-deficient hydroxyapatites, e.g., Ca_{10-X}(HPO₄)_{X}(PO₄)_{6-X}(OH)_{2-X} (0≦X≦1) (CDHA); calcium-deficient carbonate hydroxyapatites (CDCHA), and calcium-deficient carbonate apatites (CDCA);
other calcium phosphate compounds and salts known as useful in the bone graft material field, e.g., calcium polyphosphates; and calcium-, phosphate-, and/or hydroxyl-“replaced” calcium phosphates, further described below.

Calcium-replaced calcium phosphates, as used herein, are homologs of any of the above in which some of, preferably a minority of (preferably about or less than: 40%, 35%, 33.3%, 30%, 25%, 20%, 15%, or 10% of) the calciums are substituted with monovalent and/or divalent metal cation(s), e.g., sodium calcium homologs thereof, such as CaNa(PO₄);

Phosphate-replaced calcium phosphates, as used herein, are homologs of any of the above in which some of, preferably a minority of (preferably about or less than: 40%, 35%, 33.3%, 30%, 25%, 20%, 15%, or 10% of) the phosphate groups are substituted with carbonate, hydrogen phosphate, and/or silicate groups; and

Hydroxyl-replaced calcium phosphates, as used herein, are homologs of any of the above hydroxyl-containing materials in which some of, preferably a minority of (preferably about or less than: 40%, 35%, 33.3%, 30%, 25%, 20%, 15%, or 10% of) the hydroxyl groups are substituted with F, Cl, and/or I, and/or CO₃.

In one embodiment of a calcium-replaced homolog, the monovalent metal cation will be an alkali metal cation, preferably sodium; or it will be Cu(I); or a combination thereof. In one embodiment of a calcium-replaced homolog, the divalent metal cation will be an alkaline earth metal, preferably beryllium, magnesium, strontium, and/or barium, preferably magnesium, strontium, and/or barium, more preferably magnesium; in one embodiment of a calcium-replaced homolog, the divalent metal cation will be a divalent transition metal, preferably chromium, cobalt, copper, manganese, and/or zinc; or a combination thereof.

In one embodiment of a hydroxyl-replaced homolog, the halide will be fluoride, chloride, and/or iodide, preferably fluoride and/or chloride. Examples of such hydroxyl-replaced homologs include, e.g., calcium haloapatites, calcium haloahydroxypatites, and calcium halo-oxyapatites, the latter having a formula of, e.g., Ca₁₅(PO₄)₉(X)O wherein X is F, Cl, or I.

Other Matrix Components

The matrix material for a bone graft material according to the present invention may optionally contain other additives, for example: inorganic additives, including e.g., silicates, iron oxides, and the like; plasticizing agents, including lubricants and the like; binding agents, including thickeners and the like; and/or bioactive agents, including osteoinductive factors, non-osteo-specific growth factors, medicaments, osteoclast inhibitors (e.g., bisphosphonate analogs of pyrophosphate [i.e. (H₂PO₃)—CH(R)—(H₂PO₃), (H₂PO₃)—C(═R)—(H₂PO₃), or (H₂PO₃)—C(R)(R′)—(H₂PO₃)]), and the like. Preferably, the additives will collectively comprise about 10% by volume or less of the matrix, preferably about 5% or less, or about 3%, about 2%, or about 1% or less of the matrix.

In one embodiment, a bone graft material according to the present invention will include a plasticizing agent. Preferred examples of plasticizing agents include: powdered demnineralized bone matrix, preferably powdered human or bovine DBM; one or more polyether, such as a cellulose derivative, e.g., methylcellulose, carboxymethylcellulose, ethylcellulose, hydroxyethylcellulose, hydroxypropyl methylcellulose, cellulose acetate butyrate, salts thereof, and combinations thereof; and alcohols and polyols of at least three carbon atoms in length, e.g., oleyl alcohol, glycerol, sorbitol, xylitol, propylene glycol, butylene glycol, polyethylene glycol, and vinyl alcohols (polyvinylalcohols). Preferably, a cellulose derivative will be used as the plasticizer in a plasticizing-agent-containing embodiment of the present invention.

Examples of preferred bioactive agents for use in an embodiment of the present invention are: bone morphogenic proteins (BMPs), bone-derived growth factors (e.g., BDGF-2), transforming growth factors (e.g., TGF-beta), somatomedins (e.g., IGF-1), platelet-derived growth factors (PDGF), and fibroblast growth factors (FGF); general growth hormones (e.g., somatotropin) and other hormones; pharmaceuticals, e.g., anti-microbial agents, antibiotics, antiviral agents, microbistatic or virustatic agents, anti-tumor agents, and immunomodulators; and metabolism-enhancing factors, e.g., amino acids, non-hormone peptides, vitamins, and minerals; and natural extracts.

In one preferred embodiment, the matrix material will be at least substantially free of one or more of: gelatin; calcium sulfate; low molecular weight (e.g., C2-C6) esters, diols, and triols; pentaerythritol and sorbitol; synthetic biodegradable polymers, such as polyhydroxyalkanoates, e.g., PGA, PLA, and PHB polymers and copolymers; and polypeptides. In one preferred embodiment, the matrix material will be at least substantially free all of the above components, preferably about free, preferably free thereof.

Porogen Particles

In some embodiments according to the present invention, the bone graft material will comprise porogen particles in combination with the matrix material. In a preferred embodiment of a porogen particle-containing bone graft material, the composition will contain about 50% to about 90% by volume porogen particles. In a preferred embodiment, the bone graft material will comprise about 55% or more by volume porogen particles, preferably about 60% or more, about 65% or more, about 70% or more, about 75% or more, about 80% or more, or about 85% or more by volume porogen particles. In a preferred embodiment, the composition will contain about 90% or less by volume porogen particles. In a preferred embodiment, the composition will contain about 75% to about 90% porogen particles.

In a preferred embodiment, the composition will contain 80% or more by volume porogen particles, preferably more than 80%, preferably about 81% or more, or 81% or more, or more than 81%, or about 82% or more, or 82% or more, or more than 82%, or about 83% or more, or 83% or more, or more than 83%, or about 84% or more, or 84% or more, or more than 84%, or 85% or more or more than 85%. In a preferred embodiment, the composition will contain from 80% to about 90% by volume porogen particles, preferably from more than 80% to about 90% porogen particles. In a preferred embodiment, the composition will contain from about 85% to about 90% porogen particles.

Porogen particles useful herein may be made of any biocompatible, biodegradable substance that can be formed into a particle capable of at least substantially retaining its shape during processing of the bone graft material and until subjected to biodegradation-type conditions, e.g., in vivo conditions. Such substances may also be referred to herein as porogen particle materials or porogen particle “wall” materials.

The biocompatible, biodegradable substance(s) for the porogen particles may be inorganic or organic. In a preferred embodiment, the biocompatible, biodegradable substance selected will be an organic polymer, preferably a synthetic organic polymer, e.g., poly(vinyl alcohol), or a combination thereof with another polymer or a bioactive substance. Alternatively, or in addition, the organic, biocompatible, biodegradable substance will comprise demineralized bone matrix, and/or a mono-, di-, or poly-saccharide. In a preferred embodiment, the biocompatible, biodegradable substance selected will be: a calcium salt or compound; sodium chloride; or a mixture thereof; preferably a calcium phosphate or mixture thereof; or a combination of any of the foregoing comprising a bioactive substance.

In a preferred embodiment, the porogen particles will have a morphology that is any one or more of at least substantially cylindrical, at least substantially prismatic, at least substantially pyramidal, at least substantially regular polyhedral, at least substantially paraboloidal, at least substantially lenticular, at least substantially ovate, or at least substantially spherical. In a preferred embodiment, the porogen particles will include those that are at least substantially regular polyhedral, at least substantially lenticular, at least substantially ovate, or at least substantially spherical. The porogen particles may be “solid” particles, i.e. non-hollow, non-laminar particles containing the biocompatible, biodegradable substance(s); they may be hollow particles having at least one wall defining an internal “empty” space, i.e. one that is devoid of a wall material, but that may be filled with a different solid or fluid material, e.g., a bioactive substance; or they may be laminar particles having a core and at least one distinct layer, the core and layer(s) thereof being independently any wall material, the layers of the particle not defining an “empty” space, but being positioned adjacent one to the next. Hollow particles include those particles that have both laminar features and hollow space(s).

Porogen particles for use in an embodiment of the present invention preferably will have at least one dimension (i.e. axial, transverse, or lateral dimension) that is about 100 to about 500 microns. In one embodiment, all porogen particles of a given morphology will have at least one average axial, transverse, or lateral dimension that is about 100 to about 500 microns. In one embodiment, all porogen particles used in an embodiment of the present invention will independently have at least one axial, transverse, or lateral dimension that is about 100 to about 500 microns. In one embodiment, all porogen particles used in an embodiment of the present invention will collectively have at least one average axial, transverse, or lateral dimension that is about 100 to about 500 microns.

In a preferred embodiment, at least one dimension of the porogen particles will be about 100 microns or more, preferably about 120 microns or more, or about 140 microns or more. In a preferred embodiment at least one dimension of the porogen particles will be about 500 microns or less, preferably about 425 microns or less, about 350 microns or less, about 300 microns or less, or about 250 microns or less. In one preferred embodiment, the porogen particles will have at least one dimension that is about 120 to about 350 microns. In some embodiments, these gradations also apply to independent, average, and/or collective dimensions as described above. In one preferred embodiment, at least two of the axial, transverse, and lateral dimensions of the particle will independently be about 100 to about 500 microns; in one preferred embodiment, the axial, transverse, and lateral dimensions of the particle will independently be about 100 to about 500 microns.

In a preferred embodiment, the porogen particles will have a ratio of average width (lateral and transverse dimensions) to average length (main axial dimension) that is about 5:1 to about 1:5, preferably about 4:1 to about 1:4, about 3:1 to about 1:3, about 2:1 to about 1:2; in one preferred embodiment, the porogen particles will have a ratio of average width to average length that is about 1:1.

In one embodiment, the porogen particles used in the bone graft material will have at least about the same morphology. In one embodiment, the porogen particles used in the bone graft material will have at least about the same morphology and at least about the same size.

Polymers for Porogen Particles

In one embodiment of a porogen particle-containing bone graft material, the particles will comprise a biodegradable, biocompatible polymer. For purposes of the present invention, a biodegradable polymer is considered a biocompatible polymer if it is not unduly immunogenic (according to a reasonable risk-benefit analysis in sound medical judgment), and does not biodegrade to form undesirable insoluble deposits or toxic byproducts that cannot be further catabolized in vivo to form non-toxic products. Similar definitions apply for other biodegradable, biocompatible substances useful herein.

Common classes of biodegradable, biocompatible polymers useful herein include: polyesters, including polyhydroxyalkanoates, polylactones (e.g., polycaprolactones), and poly(propylene fumarates); polyanhydrides, e.g., poly(sebacic anhydride); tyrosine-derived polycarbonates (see, e.g., Muggli et al., Macromolecules 31:4120-25 (1998)); polyorthoesters; copolymers of any one or more of these with one another and/or with other biocompatible polymerizable units; and the biodegradable, biocompatible polymers described in Patent Nos. U.S. Pat. No. 6,630,155 to Chandrashekar et al. and U.S. Pat. No. 6,777,002 to Vuaridel et al.; and US Patent Publication No. 2004/0254639 to Li et al.

The monomers from which the biocompatible, biodegradable polymers useful herein are made will preferably be C1-C18 monomers, preferably C2-C12, C2-C10, C2-C8, C2-C6, or C2-C4 monomers. The polymers hereof may be homopolymers or heteropolymers of any conformation, e.g., linear, branched (including hyperbranched), cross-linked, or cyclic, etc. Useful copolymers may be statistical, random, alternating, periodic, block, or graft copolymers. By way of example, biodegradable polyhydroxyalkanoate copolymers useful herein may be, e.g., lactide, glycolide, or hydroxybutyrate copolymers synthesized with: other hydroxyacyl monomers, segments, or branches; polyalkylene oxide monomers, segments, or branches; diol or polyol monomers, segments, or branches, such as polyalkylene glycol (e.g., polyethylene or polypropylene glycol) monomers, segments, or branches; carbohydrate (including sugar alcohol, sugar acid, and other sugar derivative) monomers, segments, or branches; amino acyl monomers, segments, or branches; and/or other biocompatible polymerizable units.

Examples of preferred polyhydroxyalkanoate polymers include: poly(lactide)polymers, poly(glycolide)polymers, and poly(hydroxybutyrate)polymers, wherein the monomer units from which these are formed may have any chirality or combination of chiralities; copolymers that represent combinations of these; and copolymers that represent a combination of any of the foregoing with another hydroxyacid monomer or polymerizable monomer of another type. Examples of preferred polyhydroxyalkanoate polyester polymers include poly(glycolide), poly(L-lactide), poly(D,L-lactide), poly(L-lactide-co-glycolide), poly(L-lactide-co-D,L-lactide), poly(D,L-lactide-co-glycolide), poly(3-hydroxybutyrate), poly(3-hydroxybutyrate-co-3-hydroxyvalerate), and poly(glycolide-co-trimethylene carbonate).

The (weight average) molecular weight of biodegradable polymers typically used in bone tissue substitute materials, and which may be used in a preferred embodiment hereof, are, e.g.: about 2,000 or more, about 5,000 or more, about 10,000 or more, about 20,000 or more, about 30,000 or more, about 40,000 or more, or about 50,000 or more MW; about 100,000 or less, about 90,000 or less, about 80,000 or less, about 70,000 or less, about 60,000 or less, or about 55,000 or less MW; and about 2,000 to about 100,000 MW, more typically about 5,000 to about 100,000 MW, about 10,000 to about 90,000 MW, about 20,000 to about 80,000, about 30,000 to about 70,000, or about 40,000 to about 60,000 MW, with about 50,000 to about 55,000 MW being common. Any such molecular weight biocompatible, biodegradable polymer may be used in an embodiment of the present invention, and will be selected in conjunction with other factors that influence porogen particle in vivo degradation rates.

In vivo degradation rates for biocompatible, biodegradable polymers are discussed, e.g., in P A Gunatillake & R. Adhikari, Biodegradable synthetic polymers for tissue engineering, Eur. Cells & Mater. 5:1-16 (2003); and J C Middleton & A J Tipton, Synthetic biodegradable polymers as medical devices, Med. Plastics & Biomater. March/April 1998:30-39 (Mar 1998). In vitro degradation rates for 10 mm diameter cylindrical samples of polyhydroxyalkanoates are described in L Wu & J Ding, In vitro degradation of three-dimensional porous poly(D,L-lactide-co-glycolide) scaffolds for tissue engineering, Biomaterials 25:5821-30 (2004). Based on these data, the following estimated approximate rates of degradation may be typically expected for biodegradable polymers commonly used in bone graft materials.

TABLE 1
Typical Degradation Rates for Selected Biocompatible,
Biodegradable Polymers

| Poly(L)LA | 45 μm/wk |
|---|---|
| Poly(e-caprolactone) | 45 |
| Poly(D,L)LA | 90 |
| PGA | 140 |
| PGA-co-Me₃-carbonate | 140 |
| Copoly(D,L)L/GA 85:15 | 260 |
| Poly(propylene-fumarate) | 330 |
| Copoly(D,L)L/GA 75:25 | 520 |
| Copoly(D,L)L/GA 50:50 | 770 |

In Table 1: Poly(L)LA is poly(L-lactic acid); poly(e-caprolactone) is poly(epsilon-caprolactone); poly(D,L)LA is poly(D-,L-lactic acid); PGA is poly(glycolic acid); PGA-co-Me3-carbonate is poly(glycolic acid-co-trimethylene carbonate); copoly(D,L)L/GA 85:15, 75:25, and 50:50 are poly(D-,L-lactic acid-co-glycolic acid) polymers respectively having approximate molar proportions of 85:15, 75:25, and 50:50 lactic acid:glycolic acid monomers; and poly(propylene-fumarate) is poly(propylene glycol-co-fumaric acid).

Resorption rates, as used herein, refer to rates of resorption for individual particles that are directly in contact with biological fluid at least in part, e.g., along at one surface zone thereof. It will be understood that many uses of a bone graft material will produce an in vivo mass of bone graft material in contact with the bone, the mass of bone graft material containing both porogen particles partly embedded along a surface of the mass, and thus directly exposed to biological fluid, and porogen particles buried within the mass. Those porogen particles that are buried most distally from biological fluid sources may not be resorbed until a point in time later than that at which the original surface-exposed particle will have become resorbed, particularly in the case where the porogen particle material is or includes substance(s), such as demineralized bone matrix or other biomineralizing organic matrix materials, that are mainly resorbed by action of cells, rather than by contact with fluid alone. However, use of quickly resorbing porogen particles in the bone graft material, as taught herein, reduces the overall time until resorption of the mass' entire population of particles of a given type is complete.

Although the microns-per-week resorption rates recited in Table 1 may be typical for in vitro degradation of commonly used versions of these polymers (e.g., typically having a 50,000-55,000 MW), a variety of factors can result in different degradation rates. For example, use of a relatively lower molecular weight version of a particular polymer would be expected to increase the overall rate of degradation and dissolution of the polymer in vivo. Alternatively, use of a copolymer formed from that polymer's units with another, more hydrolysable species, e.g., a hydroxyacid and a biologically hydrolyzable carbohydrate(s) or peptide(s), would be expected to increase the rate of bulk degradation, since hydrolysis of the, e.g., carbohydrate or peptide units enhances fragmentation, resulting in lower molecular weight polymer substrates as an intermediate for degradative dissolution. Other factors and their relative effects on degradation rates for a given polymer are likewise known to one of ordinary skill in the art, e.g., polymer architecture, particle shape (geometry), particle morphology (internal structure, e.g., solid, hollow, laminar, etc.), surface area-to-volume ratio, degree of encapsulation in matrix, pH of the local in vivo environment, and accessibility of in vivo fluids and/or cells to the polymer.

Demineralized Bone Matrix

In one embodiment of a porogen particle-containing bone graft material, the particles will comprise osteoinductive demineralized bone matrix (DBM) or an osteoinductive substitute therefore; or a mixture of DBM or DBM substitute with a biodegradable polymer as described above. In a preferred embodiment, the osteoinductive demineralized bone matrix (DBM), will be at least substantially demineralized (about 90% or more), preferably about fully demineralized (about 95% or more, preferably about 97%, 98%, or 99% or more), preferably fully demineralized. The bone provided for demineralization may be cancellous and/or cortical bone, or other bony tissue, e.g., tooth tissue (e.g., dentine) or antler tissue; in a preferred embodiment, it will be cancellous and/or cortical bone. Preferably, the DBM will be prepared from bone of the species for which the bone graft material is to be used. In the case of humans, preferably the DBM will be prepared from, e.g., human, bovine, porcine, ovine, caprine, equine, cervine, piscine, or avian bone; preferably human, bovine, porcine, or ovine bone; preferably human, bovine, or porcine bone; preferably human or bovine bone; preferably human bone. In one embodiment, a DBM-containing porogen particle will contain solely DBM; in one embodiment, the DBM may be combined with at least one further substance, e.g., a biodegradable polymer or a bioactive agent or both.

Alternatively to DBM, a DBM substitute may be prepared from demineralized proteinaceous matrix obtained from another biomineralized material, preferably from another biomaterial in which the biomineralization comprises calcium compounds or salts. Examples of demineralized non-bone matrix materials include demineralized non-bony tissues, such as mollusk shells, brachiopod shells; avian shells; otoliths, otoconia; and invertebrate exoskeletons, tests, and related structures, e.g., of bryozoans, cnidarians, and echinoderms. In one preferred embodiment of a DBM substitute, demineralized mollusk or brachiopod shell will be used, preferably demineralized mollusk nacre or brachiopod semi-nacre, which comprise the inner, non-prismatic shell layer(s), whether composed of, e.g., a nacreous, crossed-lamellar, or other microstructure(s); preferably demineralized mollusk shell will be used, preferably demineralized mollusk nacre.

Demineralized bone and substitute matrix materials may be prepared by any of the methods known in the art, examples of which include treatment of the mineralized tissue, or fragments or particles thereof, with inorganic (e.g., HCl) or organic acid solutions and/or chelator(s) such as EDTA or EGTA, and other procedures, as described, e.g., in U.S. Pat. No. 6,189,537 to Wolfinbarger. The demineralized bone matrix or substitute, or the mineralized tissue from which it is prepared, may be further processed, e.g., by irradiation, sterilization, lyophilization, or any other desired useful technique known in the art.

The demineralized bone matrix so prepared may be obtained directly from the demineralization process as an osteoinductive material, i.e. retaining its native bone-growth-promoting factors. Osteoinductive factors native to such materials include, e.g.: bone morphogenetic proteins (BMPs), such as osteocalcin, osteogenin, and osteonectin. Demineralized non-bony tissue matrix materials may also provide some degree of osteoinductivity through the presence of other bioactive factors native thereto. See, e.g.: E. Lopez et al., Nacre, osteogenic and osteoinductive properties, Bull. Inst. Oceanogr. (Monaco) 14:49-58 (1993); and L. Pereira-Mouriès et al., Eur. J. Biochem. 269:4994-5003 (2002). However, preferably, a demineralized non-bony tissue matrix, where used, will be supplemented with osteoinductive factor(s). Where osteoinductive factors are added to a demineralized bone matrix or substitute, they will preferably be factors that the subject to receive the bone graft material can use to foster osteogenesis; preferably, they will be from the same species as that of the subject; or from the same individual. In the case of peptide-type factors, the term “same” includes, e.g., identity of amino acid sequence, regardless of the organism synthesizing the peptide.

An osteoinductive demineralized bone matrix (DBM) or osteoinductive substitute may be provided by supplementing a non-osteoinductive demineralized bone, or a non-osteoinductive substitute demineralized non-bony tissue matrix, with osteoinductive factors. Non-osteoinductive demineralized bone matrix is described, e.g., in U.S. Pat. No. 6,685,626 to Wironen. In a preferred embodiment, DBM or a DBM substitute retaining its native osteoinductive factors will be used. In one embodiment, DBM or a DBM substitute will be used that has been prepared by supplementing a non-osteoinductive demineralized tissue matrix with osteoinductive factors, such as BMPs, e.g., by mixing it or infusing it with, or bonding to it, such factors. Any DBM (i.e. osteoinductive DBM) or any osteoinductive DBM substitute may be further supplemented with, e.g., additional osteoinductive factors or other bioactive agents.

The osteoinductive DBM or substitute may be provided in the form of any micro or macroparticles of any morphology. Preferred formats include powders, granulates, chips, and flakes; gel formats (e.g., hydrogels, hydrogels in a carrier, such as a glycerol carrier) are also useful. Where the DBM or substitute is to be used as the porogen, it preferably will be provided in the form of particles having the porogen particle size, geometry, and morphology parameters described herein; such particles will comprise either single fragments or aggregates of the DBM and/or DBM substitute.

Compared to the preferred synthetic polymers described for porogen particles, DBM and its substitutes typically resorb at a somewhat slower rate, e.g., in some cases about 10-20 microns per week. DBM, which is typically derived from cortical bone, possesses an inherent porosity since cortical bone contains a network of approximately 20-50 micron channels (Haversian canals). Therefore, it is not necessary for DBM and DBM substitutes to resorb at the same rate as a porogen material lacking such small-diameter porosity, in order to obtain cellular penetration and calcium matrix resorption rates provided by the present invention. In one preferred embodiment in which porogen particles are DBM or a DBM substitute, these particles may have a diameter(s) in the range of 100 to about 750 microns. In an embodiment of this type, the presence of such small-diameter channels allows cellular penetration in the porogen particles while providing osteoinductive factors for cell conversion and proliferation. In another preferred embodiment, for obtaining particle DBM or DBM substitutes having particle dissolution times of about a week or less, the particles thereof will have at least one, preferably two, preferably all three of the axial, lateral, and transverse dimensions in the range of about 10 to about 100 microns, preferably about 10 to about 50, about 10 to about 40, about 10 to about 30, about 10 to about 25, or about 10 to about 20 microns. In another preferred embodiment, clusters of DBM particles in which the particles have such dimensions, preferably in the range of about 10 to about 50 microns, will be used, wherein the clusters have one, two, or three average dimensions that are from about 100 to about 500 microns. Such clusters smaller particles may also be employed for non-DBM or non-DBM substitute materials, such as synthetic polymers as described above, and is a preferred embodiment for those that resorb at rate less than 100 microns per week.

In one preferred embodiment of DBM-containing or DBM substitute-containing clusters, the smaller particles making up the cluster will be a combination of DBM or DBM substitute small particles and synthetic polymer small particles. In one preferred embodiment, porogen particles of about 100 to about 500 micron dimensions will be used that contain an admixture of a DBM or DBM substitute with one or more biocompatible, biodegradable polymer, preferably selected from those having a resorption rate of about 100 microns per week or more. In one preferred embodiment of a bone graft material comprising 100 to 500 micron DBM or DBM substitute porogen particles, both such porogen particles, and more quickly resorbing polymer porogen particles will be present within the matrix. In such an embodiment, the DBM or DBM substitute porogen particles may resorb over a period of about 6 to about 8 weeks, while the polymeric porogen particles may resorb at a rate of about 1 week or less.

Porogen Particle Additives

The material chosen for the substance of the porogen particle bulk, wall(s)/layer(s), and/or core structures may be a pure substance, as any of the polymers and copolymers, compounds, and whole (processed) tissues and tissue fragments described above, or it may be a mixture of such substances. Where a mixture is used, it may comprise any combination of the above-described porogen particle materials in any proportions. The mixture may further comprise a minority of any one or more agents that are: processing aids, such as binders (e.g., cellulose ethers) and lubricants (e.g., fatty acids); storage aids, such as preservatives and dryness-promoting agents; rehydration aids, such as wetting-facilitation agents; alginate; and the like. Preferably, such agents will constitute less than 20%, preferably about 15% or less, or about 10% or less, or about 5% or less, or about 4% or less, or about 3% or less, or about 2% or less, or about 1% or less of the mixture. Thus, the material provided for the substance of the porogen particle may be any such compound or mixture.

The porogen particles may further contain one or more added bioactive agent, either: (1) encapsulated in one or more hollow space(s) within a “hollow” particle; or (2) located within or throughout the bulk of a “solid” particle, or of a core, wall, or layer of a hollow or laminar particle. Examples of preferred bioactive agents for use in an embodiment of the present invention are: bone morphogenic proteins (e.g., BMP1-BMP15), bone-derived growth factors (e.g., BDGF-1, BDGF-2), transforming growth factors (e.g., TGF-alpha, TGF-beta), somatomedins (e.g., IGF-1, IGF-2), platelet-derived growth factors (e.g., PDGF-A, PDGF-B), fibroblast growth factors (e.g., αFGF, βFGF), osteoblast stimulating factors (e.g., OSF-1, OSF-2), and sonic hedgehog protein (SHH); other hormones, growth factors, and differentiation factors (e.g., somatotropin, epidermal growth factor, vascular-endothelial growth factor; osteopontin, bone sialoprotein, α2HS-glycoprotein; parathyroidhormone-related protein, cementum-derived growth factor); biogenic proteins and tissue preparations (e.g., collagen, carbohydrates, cartilage); gene therapy agents, including naked or carrier-associated nucleic acids (e.g., single- or multi-gene constructs either alone or attached to further moieties, such as constructs contained within a plasmid, viral, or other vector), examples of which include nucleic acids encoding bone-growth-promoting polypeptides or their precursors, e.g., sonic hedgehog protein (see, e.g., P C Edwards et al., Gene Ther. 12:75-86 (2005)), BMPs (see, e.g., C A Dunn et al., Molec. Ther. 11(2):294-99 (2005)), Runx2, or peptide hormones, or anti-sense nucleic acids and nucleic acid analogs, e.g., for inhibiting expression of bone-degradation-promoting factors; pharmaceuticals, e.g., anti-microbial agents, antibiotics, antiviral agents, microbistatic or virustatic agents, anti-tumor agents, and immunomodulators; and metabolism-enhancing factors, e.g., amino acids, non-hormone peptides, vitamins, minerals, and natural extracts (e.g., botanical extracts). The bioactive agent preparation may itself contain a minority of, e.g., processing, preserving, or hydration enhancing agents. Such bioactive agents or bioactive agent preparations may be used in either the porogen particle(s) or the calcium matrix material, or both. Where both contain bioactive agent(s), the agent(s) may be the same or different.

In some embodiments, a plurality of different porogen particles may be used, which can differ in any desired ways, e.g., in size, morphology, bulk material, bioactive agent(s), and/or other additives. Porogen particles having the dimensions and characteristics described herein may also be used in combination with “other porogens” that can resorb at a different rate or rates, or that may be of a different size (e.g., nanoparticles) or morphology (e.g., fibrous or filamentous) than the “porogen particles” described herein. Examples of such uses include the use of polymer “porogen particles” along with slower-resorbing DBM particles or with DBM small-particle clusters. Thus, a bone graft material according to the present invention may comprise a combination of “porogen particles” as defined herein, with “other porogens” known in the art. In a preferred embodiment, at least half, preferably at least a majority of the porogens in a bond graft material according to the present invention will be “porogen particles” having the characteristics as defined herein. In a preferred embodiment, about 60% or more, about 70% or more, about 75% or more, about 80% or more, about 85% or more, about 90% or more, about 95% or more, or about 98% or more of the total volume of porogens in the composition will be comprised of “porogen particles” as defined herein. Preferably at least substantially about all, preferably about all, preferably all of the porogens in a composition according to the present invention will be “porogen particles” as defined herein.

In a preferred embodiment in which one or more bioactive agent preparation is included in the bulk of a solid particle or core, wall, or layer of a hollow or laminar particle, the additive(s) will make up about 10% or less by volume of the material, preferably about 5% or less, about 4% or less, about 3% or less, about 2% or less, or about 1% or less. The maximal amount of bioactive agent preparation included in a space in a hollow particle can be determined by the volume of the space. The format for additives to be included in porogen particles according to the present invention will be powders, particles, or solutions of any morphology or consistency (e.g., dry, paste, or slurry), provided that the additives can be effectively incorporated into either the bulk substance of the porogen particle or into a void within.

Porogen Particle Parameter Selection

Regardless of the formulation of the biocompatible material selected for a porogen particle, e.g., whatever the identity of a biocompatible biodegradable polymer selected, for use in a given embodiment of the present invention, any of the techniques described in the above-cited references, in the articles cited therein, and in other references known in the art, may be used to obtain approximate biodegradation rates therefor, whether relative or absolute. These rates can then be used to select a dimension for a particular geometry or morphology desired for in vivo biodegradation over a selected time period. For example, where degradation is desired over a period of 3 days, and the desired geometry-plus-morphology is a substantially spherical “solid” microparticle partly embedded in a ceramic- or glass-type matrix and having at least one exposed surface, the particle diameter could be about 220 microns for a polymer that degrades at a rate of about 520 microns per week. Likewise, where a 3-day degradation period is desired for a similarly situated, single-walled hollow microparticle, the wall thickness could be about 20 microns for a polymer that degrades at a rate of about 45 microns per week.

In a preferred embodiment according to the present invention, the biocompatible, biodegradable polymer or DBM or DBM substitute to be used, will be selected in light of other biodegradation-rate influencing factors, to obtain either: porogen particles that contain the polymer, DBM or substitute, or of a polymer-, DBM-, or DBM substitute-bioactive ingredient combination throughout the bulk of the particle (i.e. are neither hollow nor laminar), and which can be biodegraded in vivo within about 10 minutes to about 7 days; or porogen particles that are hollow or laminar particles having at least one wall or layer that is made of the polymer or solid polymer-bioactive ingredient combination, at least one wall of which can be biodegraded in vivo within about 10 minutes to about 7 days, i.e. that average time to dissolution in vivo for the particle or wall is a value within that range. In a preferred embodiment, the polymer or polymeric combination will be selected in conjunction with other particle parameters to obtain porogens in which average time to dissolution in vivo for the particle or wall is within about 10 minutes to about 5 days, or about 10 minutes to about 3 days.

Preparation of the Bone Graft Material

The matrix material and porogen particle components, and other optional components, selected for a bone graft material according to the present invention will be combined in any order. In a preferred embodiment, the matrix material and porogen particle components will be pre-mixed, with optional inclusion of other matrix additives; and then a bioactive substance(s) will be combined therewith. Alternatively, a bioactive substance(s) may be optionally included in the matrix material(s) and/or in the porogen particles before they are combined.

The bone graft material, where provided in a hydratable form, e.g., a dry powdered or granulated form or a dry solid block or plug form or any semi-solid form, may be wetted or further wetted with a wetting agent to produce a wetted format, such as a paste, putty, or pre-wetted solid for administration to a subject. In one embodiment of a wetted composition, the liquid used for wetting will be a neat solution or a biological fluid. Where a neat solution is used, it will preferably be an aqueous saline or a buffered aqueous solution, such as phosphate-buffered saline or a cell growth medium, having a biocompatible pH (e.g., about pH6 to about pH8, preferably about pH 6.5 to about pH 7.5); the biocompatible pH may be inherent to the wetting liquid before use, or may be a result of applying the liquid to the composition. Where a biological fluid is used, it will be biocompatible with the subject to be treated with the bone graft material, e.g., not unduly immunogenic or toxic to the individual to receive it, in accordance with a reasonable risk-benefit ratio assessed in sound medical judgment. In one embodiment, the biological fluid will be autologous to the patient to be treated.

Useful biological fluids from complex animals and humans may be vascular or extra-vascular. Examples of such biological fluid wetting agents include, but are not limited to: blood, serum, platelet concentrate, bone marrow aspirate, and synovial fluid. A biological fluid may be used in the form obtained from the biological source, or it may be processed by application of one ore more desired useful techniques, examples of which include, separation techniques, such as filtration (macro-, micro-, or ultra-filtration); purification techniques, such as dialysis; concentration techniques; and sterilization techniques.

The neat solution or biological fluid may further be supplemented with one or more additives. Examples of additives include, but are not limited to: medicaments; polypeptides, including enzymes, proteins, and proteinaceous tissue preparations; peptide hormones and growth factors; non-peptide hormones and growth factors; vitamins; minerals; and the like.

The bone graft material, or components thereof, may be treated to contain or harbor, internally or externally, living cells. The cells may be subject-autologous cells, subject-matched donor cells, or subject-compatible cultured cells. Example of such cells include, e.g.: osteoblasts; pluripotent stem cells, such as osteoblast precursors (e.g., adipose tissue-derived and bone-marrow derived stem cells); and totipotent stem cells. In a preferred cell-containing embodiment these will be applied to the bone graft material by suffusing it with a neat solution or biological fluid containing such cells.

Commercial Packages

A commercial package may provide a bone graft material according to the present invention as a pre-moistened paste or other semi-solid or liquid formulation; or it may provide the bone graft material as a dry powder or solid. Where the bone graft material is supplied in a dry form, an aqueous solution may be provided in the commercial package for use in wetting the dry material. For example, an ionic solution, such as saline, preferably a buffered solution, such as phosphate-buffered saline, will be provided. A commercial package will contain instructions for use, and optionally for further preparation of the bone graft material prior to use. The commercial package may optionally contain a device or devices for use in mixing, shaping, and/or administering (e.g,. inserting, injecting, or applying) the bone graft material.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A bone graft material having a calcium matrix component and porogen particles, present in a porogen particle-to-matrix material ratio of 1:1 to about 9:1.

2. The bone graft material according to claim 1, wherein said calcium matrix component comprises calcium sodium phosphate, tricalcium phosphate, dicalcium phosphate dihydrate, calcium hydroxyapatite, or a combination thereof.

3. The bone graft material according to claim 1, wherein said calcium matrix component further comprises at least one plasticizing agent, at least one bioactive agent, or a combination thereof.

4. The bone graft material according to claim 1, wherein said porogen particles comprise at least one biocompatible, biodegradable substance.

5. The bone graft material according to claim 4, wherein said biocompatible, biodegradable substance is osteoinductive demineralized bone matrix (DBM) or an osteoinductive DBM substitute.

6. The bone graft material according to claim 5, wherein said DBM is human or bovine DBM and said DBM substitute is osteoinductive-factor-supplemented demineralized mollusk nacre.

7. The bone graft material according to claim 4, wherein said biocompatible, biodegradable substance is a biocompatible, biodegradable polymer.

8. The bone graft material according to claim 7, wherein said biocompatible, biodegradable polymer is a biocompatible, biodegradable polyester, polyanhydride, tyrosine-derived polycarbonate, polyorthoester, or polymer blend containing at least one such polymer.

9. The bone graft material according to claim 7, wherein said biocompatible, biodegradable polymer is a biocompatible, biodegradable polyester.

10. The bone graft material according to claim 9, wherein said biocompatible, biodegradable polyester is a biocompatible, biodegradable polyhydroxyalkanoate.

11. The bone graft material according to claim 10, wherein said biocompatible, biodegradable polyhydroxyalkanoate is formed from hydroxyl-carboxylic acid monomers independently containing about 2 to about 6 carbon atoms, from lactone forms thereof, or from a combination thereof.

12. The bone graft material according to claim 11, wherein the average size of said carboxylic acid monomers is about 2 to about 4 carbon atoms.

13. The bone graft material according to claim 7, wherein said biocompatible, biodegradable polymer has a weight average molecular weight of about 2,000 to about 100,000.

14. The bone graft material according to claim 7, wherein said biocompatible, biodegradable polymer has a weight average molecular weight of about 20,000 to about 80,000.

15. The bone graft material according to claim 7, wherein said biocompatible, biodegradable polymer has a weight average molecular weight of about 50,000 to about 55,000.

16. The bone graft material according to claim 1, wherein said porogen particles comprise at least one bioactive agent.

17. The bone graft material according to claim 1, wherein said porogen particles comprise at least substantially regular polyhedral, lenticular, ovate, or spherical particles, or a combination thereof.

18. The bone graft material according to claim 1, wherein said porogen particles collectively have at least one average axial, transverse, or lateral dimension that is about 100 to about 500 microns.

19. The bone graft material according to claim 1, wherein said porogen particles collectively have at least one average axial, transverse, or lateral dimension that is about 120 to about 425 microns.

20. The bone graft material according to claim 1, wherein said porogen particles collectively have at least one average axial, transverse, or lateral dimension that is about 120 to about 350 microns.

21. The bone graft material according to claim 1, wherein each of the average axial, transverse, and lateral dimensions of the porogen particles is independently about 100 to about 500 microns.

22. The bone graft material according to claim 1, wherein each of the average axial, transverse, and lateral dimensions of the porogen particles is independently about 120 to about 425 microns.

23. The bone graft material according to claim 1, wherein each of the average axial, transverse, and lateral dimensions of the porogen particles is independently about 120 to about 350 microns.

24. The bone graft material according to claim 1, wherein said porogen particles have a ratio of average width to average length that is from about 5:1 to about 1:5.

25. The bone graft material according to claim 1, wherein said porogen particles have a ratio of average width to average length that is from about 2:1 to about 1:2.

26. The bone graft material according to claim 1, wherein said porogen particles have a ratio of average width to average length that is about 1:1.

27. The bone graft material according to claim 1, wherein said porogen particles make up about 50% to about 90% by volume of the bone graft material.

28. The bone graft material according to claim 1, wherein said porogen particles make up about 75% to about 90% by volume of the bone graft material.

29. The bone graft material according to claim 1, wherein said porogen particles make up about 80% to about 90% by volume of the bone graft material.

30. The bone graft material according to claim 1, wherein said porogen particles make up more than 80% to about 90% by volume of the bone graft material.

31. The bone graft material according to claim 1, wherein said porogen particles are solid particles.

32. The bone graft material according to claim 1, wherein said porogen particles are capable of being biodegraded in vivo in about 10 minutes to about 7 days, or wherein said porogen particles are DBM or DBM substitute porogen particles that are capable of being biodegraded in vivo in about 10 minutes to about 8 weeks, or said porogen particles comprise a combination of these porogen particles.

33. The bone graft material according to claim 1, wherein said porogen particles are hollow or laminar particles.

34. The bone graft material according to claim 33, wherein at least one wall or layer of said porogen particles is capable of being biodegraded in about 10 minutes to about 7 days, or wherein at least one wall or layer of said porogen particles is DBM or a DBM substitute wall or layer that is capable of being biodegraded in vivo in about 10 minutes to about 8 weeks, or said porogen particles comprise a combination of these hollow or laminar particles.

35. The bone graft material according to of claim 1, wherein said material is provided in the form of a paste, injectible solution or slurry, dry powder, or dry solid.

36. The bone graft material according to claim 1, wherein said material is provided in the form of a paste, injectible solution or slurry that has been hydrated by application of a neat solution or biological fluid to a dry bone graft material according to claim 1.

37. A commercial package containing a bone graft material according to claim 1 and instructions for use thereof in repairing bone.

38. A method for repairing bone comprising:
providing a bone graft material according to claim 1, and
administering said bone graft material to a living bone tissue surface in need thereof.

39. The method according to claim 38, said method further comprising permitting said material to remain at an in vivo site in which it is placed, for a sufficient time to permit the porogen particles thereof to be biodegraded in vivo.

40. The bone graft material according to claim 1, wherein said porogen particles include a plurality of first porogen particles comprising osteoinductive DBM or osteoinductive DBM substitute, and a plurality of second porogen particles comprising a biocompatible, biodegradable polymer or polymers.

41. The bone graft material according to of claim 30, wherein said material is provided in the form of a paste, injectible solution or slurry, dry powder, or dry solid.

42. The bone graft material according to claim 30, wherein said material is provided in the form of a paste, injectible solution or slurry that has been hydrated by application of a neat solution or biological fluid to a dry bone graft material according to claim 30.

43. A commercial package containing a bone graft material according to claim 30 and instructions for use thereof in repairing bone.

44. A method for repairing bone comprising:
providing a bone graft material according to claim 30, and
administering said bone graft material to a living bone tissue surface in need thereof.
